(19) United States
(12) Patent Application Publication
(10) Pub. No.: US 2011/0245607 A1
(43) Pub. Date: Oct. 6, 2011

(54) ENDOSCOPIC GASEOUS MATERIAL FEED SYSTEM

(76) Inventors: Kentaro HAYASHI, Kanagawa (JP); Shozo Iyama, Kanagawa (JP)

(21) Appl. No.: 13/016,163

(22) Filed: Jan. 28, 2011

(30) Foreign Application Priority Data
Mar. 31, 2010 (JP) 2010-082659

(51) Int. Cl. A61B 1/04

(52) U.S. Cl. 600/109

(57) ABSTRACT

An endoscopic gaseous material feed system comprises an endoscope 1 having a solid-state image sensor device 81 incorporated into a fore distal end portion of an elongated endoscopic insertion rod to be introduced into a body cavity of a patient or examinee, a gas feeder 3 loaded with a gas tank 60 which is packed with carbon dioxide gas to be sent to the body cavity, a light source 2 incorporating an air pump to supply air to the body cavity, a processor 4 for generating video signal data of an endoscopically captured picture image on the basis of electric signals from the solid-state image sensor 81, a detector 87 provided in the processor 4 and adapted to detect whether or not the endoscope is currently placed in a body cavity, from video signal data of the processor, and a feed gas source controller 85 provided in the processor and adapted to permit a supply of carbon dioxide gas or air when the detector 87 makes an affirmative judgment that the endoscope 1 is currently placed in a body cavity and to prohibit a supply of carbon dioxide gas when the detector 87 makes a negative judgment.

### TECHNICAL FIELD OF THE INVENTION

This invention relates to an endoscopic gaseous material feed system for medical use, including a gas feeder to supply carbon dioxide gas to an endoscope connected to a light source with an air supply source.

### BACKGROUND OF THE INVENTION

In endoscopy, air or a gas, as a gaseous material, is supplied to a body cavity of a patient or examinee for the purpose of securing a view field of an endoscope or for securing a space which is required for manipulation of a surgical or biopsy tool as an unlimited source of gaseous material. Heretofore, it has been the general practice to use air as a gaseous material to be delivered to a body cavity. However, instead of air, carbon dioxide gas (CO₂), as another gaseous material, is increasingly used for this purpose, in consideration of better in vivo absorption and less damages to a patient or examinee.

Air is supplied by an air pump which is incorporated into a light source, while carbon dioxide gas is supplied by the use of a gas feeder. More specifically, carbon dioxide gas is supplied from a gas tank which is mounted on the gas feeder and packed with carbon dioxide gas to be consumed. A light source and a gas feeder of this sort are disclosed in Japanese Laid-Open Patent Application 2006-43130. In this prior art application, for the purpose of preventing wasteful consumption of carbon dioxide gas, arrangements are made to close a valve unit of a gas feeder when neither endoscopic examination nor treatment is underway.

In Japanese Laid-Open Patent Application 2006-43130 mentioned above, suspension of a carbon dioxide gas supply triggered on the basis of criteria such as turn-off a light source, total amount of gas feed, feed gas pressure, stoppage of operation of a second CCU which generates endoscopic picture images, flexure or non-flexure of articularly flexing section of an endoscopic insertion rod. These criteria indicate operating conditions of the endoscope or gas feeder, which can be relied on in assuming whether or not an endoscopic examination or treatment is currently underway. However, in this case, a feed gas controller cannot detect the actual status of the endoscope, i.e. whether or not the endoscope is currently placed in a body cavity.

Accordingly, the assumptive judgments can differ from actual conditions, and a gas supply can be suspended while an endoscope is still placed in a body cavity. A gas supply should always be feasible as long as an endoscope is placed in a body cavity, so that it is important to prevent total shutdown of gas supply by a misjudgment. This should be considered more preferentially over the problem of wasteful consumption of carbon dioxide gas.

For instance, in the technology of Japanese Laid-Open Patent Application 2006-43130, a judgment for suspension of a gas supply is made on the basis of criteria such as a turn-off action on the light source and stoppage of a second CCU. However, these actions simply indicate an action of turning off a power switch on the light source and have nothing to do with the current status of the endoscope whether or not it is currently placed in a body cavity for an endoscopic examination or treatment. The light source is turned off basically after extraction of the endoscope, but actually the turn-off actions is not linked with the extraction of the endoscope. Therefore, there is a possibility of a gas supply being shut down when an endoscope is placed in a body cavity. Further, a supply carbon dioxide gas is kept on until a power switch is turned off. That is to say, when neither an endoscopic examination nor treatment is underway, carbon dioxide gas is wastefully consumed as long as the power switch is on.

In case the gas supply is controlled on the basis of criteria such as total amount of gas feed and pressure of feed gas or on the basis of flexures of the articularly flexing section of the endoscope, judgments are made by a feed gas controller irrespective of extraction of the endoscope. For example, a judgment is made to suspend a gas supply when the articularly flexing section is left in the same posture for a predetermined time period without being flexed. In this case, a gas supply is suspended even if the endoscope is placed in a body cavity. On the other and, a surveillance time on the flexing section is elongated in order to avoid situations like this, carbon dioxide gas can be wastefully supplied for a long time through an endoscope which is not placed in a body cavity.

### SUMMARY OF THE INVENTION

With the foregoing in view, it is an object of the present invention to provide an endoscopic gas feed system which is adapted to prevent shutdown of a gas supply while an endoscope is placed in a body cavity of a patient or examinee, while suppressing wasteful consumption of carbon dioxide gas.

In order to achieve the above-stated objective, according to the present invention, there is provided an endoscopic gas feed system which comprises: an endoscope having a solid-state image sensor device incorporated into a fore distal end portion of an elongated insertion rod to be placed in a body cavity of a patient or examinee; a gas feeder having a gas tank packed with carbon dioxide gas to be supplied to the body cavity, a light source incorporating an air pump to supply air to the body cavity; a processor adapted to generate video signal data of endoscopically captured picture images on the basis electric signals from the solid-state image sensor; a detector adapted to judge whether or not the endoscope is currently placed in a body cavity, on the basis of video signal data from the processor; and a feed gas source controller adapted to permit a supply of carbon dioxide gas or air when the detector makes an affirmative judgment that the endoscope is currently placed in a body cavity and to prohibit a supply of carbon dioxide gas when the detector makes a negative judgment.

According to this endoscopic gas feed system, whether or not an endoscope is currently placed in a body cavity is judged on the basis of video signal data produced by the processor, and a supply of a gas or air, as a gaseous material, through the endoscope is controlled according to the results of the judgment. The video signal data indicate the current status of the endoscope, so that a supply of carbon dioxide gas is permitted or suspended depending upon the video signal data which indicate that the endoscope is or is not placed in a body cavity. Therefore, a supply of the gas is invariably permitted and not suspended whenever an endoscope is placed in a body cavity. On the other hand, a supply of carbon dioxide gas is suspended when the endoscope is not in a body cavity, avoiding wasteful consumption of carbon dioxide gas.

In a preferred form of the invention, the detector includes a color tone detector which is adapted to make an affirmative judgment that an endoscope is currently placed in a body cavity, upon detection of a reddish color tone in video signal data, otherwise making a negative judgment.

In this case, whether or not an endoscope is currently placed in a body cavity is judged by existence or non-existence of a reddish color tone in video signal data. Picture images in an endoscopy room differ in color tone quite clearly from picture images in a body cavity. Therefore, a judgment whether or not in a body cavity can be made by checking for a reddish color tone or tones in video signal data. This judgment can be made automatically, without necessitating a visual check on the part of an operator.

In a preferred form of the invention, the detector includes a motion detector which is adapted to check for a motion of a subject on the basis of video signal data of endoscopic picture images and, upon detection of a motion, to make an affirmative judgment that an endoscope is currently placed in a body cavity, and otherwise to make a negative judgment.

In this case, the status of an endoscope, whether or not it is placed in a body cavity, is judged through detection of a motion of a subject. For example, a motion incessantly occurs to an intracavitary wall portion under the influence of peristalsis. Therefore, by detection of a motion, it is recognized that an endoscope is currently placed in a body cavity. Thus, it is possible to judge the current status of an endoscope automatically.

In still another preferred form of the invention, the detector includes a motion detector which is adapted to check for a motion of a subject and, upon detection of a motion, to make an affirmative judgment that an endoscope is currently placed in a body cavity, otherwise making a negative judgment, and the feed gas source controller is adapted to control said gas feeder to supply carbon dioxide gas when an affirmative judgment is issued from both of the color tone detector and the motion detector.

In this case, the status of an endoscope is checked by both of the color tone detector and the motion detector. Detection of a reddish color tone in addition to a motion of a subject, it becomes possible to judge in a more assured manner that the endoscope is currently placed in a body cavity. Namely, in this case, it becomes possible to make a more accurate judgment by the use of two detectors.

In another preferred form of the invention, the feed gas source controller is adapted to control the light source to supply air when an affirmative judgment is issued from only one of the color tone detector and the motion detector.

In this case, air is supplied instead of carbon dioxide gas when different judgments are made by the color tone detector and the motion detector. Under these circumstances, it is difficult to ascertain whether or not an endoscope is currently placed in a body cavity, so air is supplied through the endoscope without totally suspending a gas supply. Since the source of air is unlimited, air can be supplied free of the problem of wasteful carbon dioxide gas consumption even if the endoscope is not placed in a body cavity at this time.

Further, in still another preferred form of the invention, the color tone detector is adapted to carry out a two-stage checkup of video signal data, detecting a reddish color tone in video signal data in the first stage and checking for a red color level or strength in a second stage, and the feed gas source controller is adapted to control the gas feeder to supply carbon dioxide gas through the endoscope when a reddish color tone of a strong level is detected, and to control the light source to supply air through the endoscope when a reddish color tone of a relatively weak level is detected.

According to this endoscopic gas feed system, a reddish color tone in video signal data is detected by a two-stage checkup. Carbon dioxide gas is supplied through the endoscope upon detection of a reddish color tone of a strong level which is invariably spotted when an endoscope is placed in a body cavity. Detection of a reddish color tone of a weak level may or may not mean that an endoscope is currently placed in a body cavity. Therefore, in this case, the light source is controlled to supply air which has an unlimitedly abundant source, avoiding to totally suspend a gas supply in such a way as to contribute to suppression of wasteful carbon dioxide gas consumption.

As described above, according to the present invention, whether or not an endoscope is currently placed in a body cavity detected from video signal data of endoscopic picture images to make a rational feed gas control depending upon the current status of the endoscope. Accordingly, it becomes possible to directly grip the current status of an endoscope, permitting a carbon dioxide gas supply as long as it is placed in a body cavity and suspending the supply of carbon dioxide gas as soon as the endoscope is extracted out of the body cavity to suppress wasteful consumption of carbon dioxide gas.

### BRIEF DESCRIPTION OF THE DRAWINGS

In the accompanying drawings:

FIG. 1 is a schematic illustration of an endoscopic gaseous material feed system as a whole;

FIG. 2 is a block diagram, showing connections of various components of the gaseous material feed system; and

FIG. 3 is a block diagram showing a general configuration of a processor.

### DESCRIPTION OF PREFERRED EMBODIMENTS

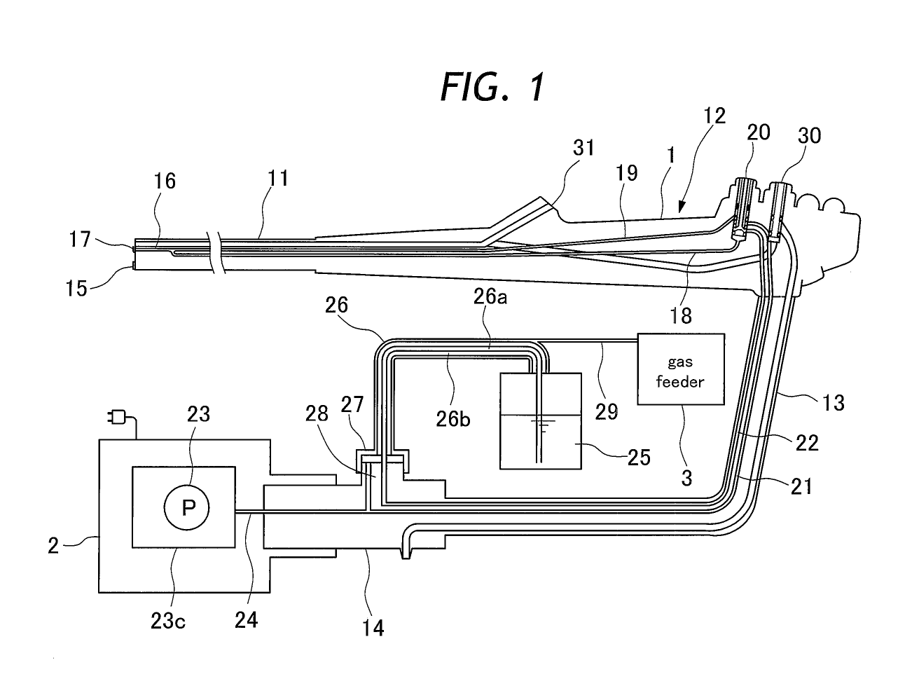

Hereafter, the present invention is described more particularly by way of its preferred embodiments. Needless to say, the present invention should not be construed as being limited to the particular forms shown. Schematically shown in FIG. 1 is a general layout of an endoscopic gaseous material feed system according to the invention, largely constituted by an endoscope 1, a light source 2, a gas feeder 3 and a processor 4 as described in greater detail hereinafter. The endoscope 1 is adapted to be introduced into a body cavity of a patient or examinee at the time of an endoscopic medical treatment or examination. The gaseous material feed system of the invention is applicable not only to flexible type endoscopes in use for upper or lower endoscopy but also to rigid type endoscopes like a laparoscope. The endoscope 1 is resorted to for a medical treatment, for example, for an endoscopic surgical operation. The light source 1, a source of illumination light, incorporates an air pump 23 to supply air to a body cavity through the endoscope 1. The gas feeder 3 is a source of carbon dioxide gas to be supplied to a body cavity. The processor 4 performs video signal processing operations to generate picture images captured through the endoscope 1. As shown in FIG. 1, the light source 2 and the gas feeder 4 are provided as separate units, but may be provided as one complex unit if desired.

The endoscope 1 is largely constituted by an elongated insertion rod 11, a manipulating head assembly 12 and a universal cable 13. The insertion rod 11 is introduced into a body cavity of a patient or examinee at the time of an endoscopic treatment or examination, and connected to operating mechanisms on the manipulating head assembly 12 which is gripped by an operator. Provided at a proximal end of the universal cable 13 is a light source connector 14 which is disconnectibly connectable to the light source 2. Although not particularly shown in the drawing, an illumination window and an endoscopic observation window 15 are provided at the fore distal end of the endoscopic insertion rod 11. Picture images of a subject (e.g. an intracavitary site), under illumination light from the illumination window, are captured through the observation window 15. Fitted in the observation window 15 is a camera unit including an objective lens and a solid-state image sensor device 81, which will be described hereinafter.

The endoscope 1 is internally provided with a fluid supply mechanism. When the observation window 15 is contaminated with body fluids in a body cavity, a liquid is supplied through the endoscope to wash away the contaminants. A liquid is also supplied to clean and to get clearer images of an intracavitary wall under examination, or for the purpose of irrigative cleaning. On the other hand, a gas is supplied to inflate a body cavity or to dissipate droplets of a cleaning liquid from the observation window 15 after a washing operation. Endoscopes in general are adapted to deliver water or air through such a fluid supply mechanism.

The above-mentioned fluid supply mechanism includes a fluid supply passage 16 internally through the insertion rod 11, the fore distal end of the fluid supply passage 16 being formed into the shape of a jet nozzle 17 which is directed toward the observation window 15. The fluid supply passage 16 is bifurcated into a gas supply passage 18 and a liquid supply passage 19 in the course of the insertion rod 2 and extended into the manipulating head assembly 12. The gas supply passage 18 and liquid supply passage 19 are connected to a fluid feed valve 20 which is provided on the manipulating head assembly 20. The fluid feed valve 20 is connected with a gas feed passage 21 and a liquid feed passage 22 which can be brought into and out of communication by operating the fluid feed valve 20.

A gas supply route is constituted by the gas supply passage 18 and the gas feed passage 21, while a liquid supply route is constituted by the liquid supply passage 19 and the liquid feed passage 22. The fluid supply passage 16 is commonly used as a compressed air supply conduit and a cleaning liquid supply conduit, and compressed air or a cleaning liquid is selectively supplied to the jet nozzle 17. The gas feed passage 21 and liquid feed passage 22 are extended into the universal cable 13 from the manipulating head assembly 12 and led to the light source connector 14.

An illumination lamp (not shown) is provided in the light source 2 to serve as an illumination light source. Illumination light from the lamp is transferred by a light guide which is extended toward an endoscopic observation means at the fore distal end of the insertion rod 11, and projected through the above-mentioned illumination window. An electric connector (not shown) which is branched out from the universal cable 13 is disconnectibly connected to the processor 4.

An air pump 23 is built in a pump control block 23c of the light source 2 to deliver compressed air to the gas supply passage 18, and air is fed under pressure from the air pump 23 to a compressed air pipe 24. The liquid supply passage 19 serves as a supply route for a cleaning liquid which comes from a liquid feed tank 25, located outside the light source 2. Connected to the liquid feed tank 25 is a dual flexible tube 26 consisting of an inner tube serving as a cleaning liquid conduit 26a and an outer tube serving as a pressurization conduit 26b. A piping connection member 27 at the fore distal end of the dual flexible tube 26 is disconnectibly connected to a piping connection member 28 which is provided on the light source connector 14. One end of the cleaning liquid conduit tube 26a of the dual flexible tube 26 is immersed in a cleaning liquid in the liquid feed tank 25, while one end of the pressurization conduit tube 26b is located in an exposed state above a liquid surface in the tank 25. A feed gas conduit 29 is connected to the gas feeder 3 as a carbon dioxide gas supply route, and also connected to the pressurization conduit 26b. Thus, the pressurization conduit 26b is bifurcated at a proximal end, one of the bifurcated conduits serving as the feed gas conduit 29 and the other one being led to the feed liquid tank 25. The pressurization conduit 26b is connected to the feed gas conduit 29 and at the same time to a compressed air conduit 24 from the air pump 23 within the light source connector 14. Accordingly, a pressure is applied to the surface of the cleaning liquid in the liquid feed tank 25 by sending carbon dioxide gas and air through the pressurization conduit 26b which is exposed and opened to an upper portion of the liquid feed tank 25. Further, in addition to the fluid feed valve 20 is provided on the manipulating head assembly 12, along with a suction valve 30 and an entrance 31 for introduction of a surgical or biopsy tool.

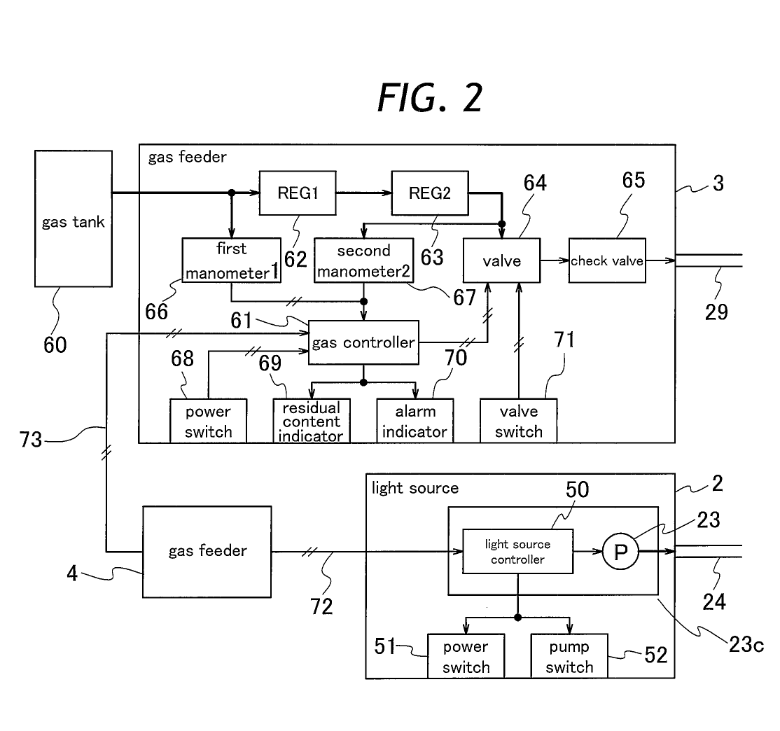

Schematically shown in FIG. 2 are general configurations of the light source 2 and the gas feeder 3. The light source 2 is largely comprised of the above-mentioned air pump 23 and a light source controller 50, a power switch 51 and a pump switch 52. By pumping action of the air pump 23, compressed air is delivered to the compressed air conduit 24. The light source controller 50 is provided to activate or deactivate (stop) the air motor 23.

The power switch 51 serves as a selector switch for turning on or off the light source 2. When this switch 51 is off, the air pump 23 as well as supply of illumination light is turned off. The pump switch 52 is a switch for putting the air pump 23 in operation. In the case of the particular embodiment shown, the light source controller 50 is arranged to start the air pump 23 automatically when the power switch 51 is turned on. However, if desired, arrangements may be made to turn on the air motor 23 manually by way of the pump switch 52 instead of starting same automatically as soon as the power switch 51 is turned on.

A gas tank 60 is replaceably mounted on the gas feeder 3, which is largely constituted by a gas controller 61, first regulator (REG1) 62, second regulator (REG2) 63, valve 64, check valve 65, first manometer (manometer 1) 66, second manometer (manometer 2) 67, power switch 68, residual content indicator 69, alarm indicator 70 and a valve switch 71. The gas tank 60 is a carbon dioxide tank which is packed with carbon dioxide gas. Carbon dioxide gas in the gas tank 60 is consumed when in use, and can be dismantled from the gas feeder 3 and replaced when it is totally consumed or with appropriate timing.

The gas controller 61 is at the control of the gas feeder 3 as a whole. The first regulator 62 is connected to the gas tank 60 to reduce the pressure of carbon dioxide gas from the gas tank 60. Since carbon dioxide gas from the gas tank 60 is at a high pressure, it is regulated to a suitable level by two-stage pressure reduction. That is to say, after a pressure reduction by the first regulator 62, the pressure of carbon dioxide gas is further reduced by the second regulator 63 in the second stage. The valve 64 is interposed between the second regulator 63 and the check valve 65 to establish or cut communication with the feed gas conduit 29 by on-off control. As for the valve 64, for example, there may be employed an electromagnetic valve, energizing and de-energizing a solenoid for the on-off control. The check valve 65 is provided to prevent an inverse flow of a gas from the side of the feed gas conduit 29.

The first manometer 66 serves to detect the pressure of carbon dioxide gas in the gas tank 60, while the second manometer 67 serves to detect the pressure of carbon dioxide gas after reduction by the second regulator 63. The results of detection are output to the gas controller 61 thereby to recognize the residual quantity of carbon dioxide gas. The power switch 68 is provided to turn on and off the power supply to the gas feeder 3. The residual content indicator 69 is adapted to indicate a residual quantity of carbon dioxide gas as recognized by the gas controller 61. The alarm indicator 70 is adapted to indicate an alarm sign when an abnormal gas pressure is detected by either the first manometer 66 or second manometer 67. The alarm indicator 70 may be arranged to give off an alarm sound if desired. The valve switch 71 is connected to the valve 64 for on-off control of the latter. The valve 64 is basically opened and closed by the gas controller 61, but can be manually opened and closed by means of the valve switch 71.

The processor 4 is connected with the endoscope 1, the light source 2 and the gas feeder 3. More particularly, the processor 4 is connected with the endoscope 1, light source 2 and gas feeder 3 by way of universal cable 13, first communication line 72 and second communication line 73, respectively. Namely, the light source 2 and gas feeder 3 are connected with each other by way of the processor 4, which is at the control of the light source 2 and the gas feeder 3.

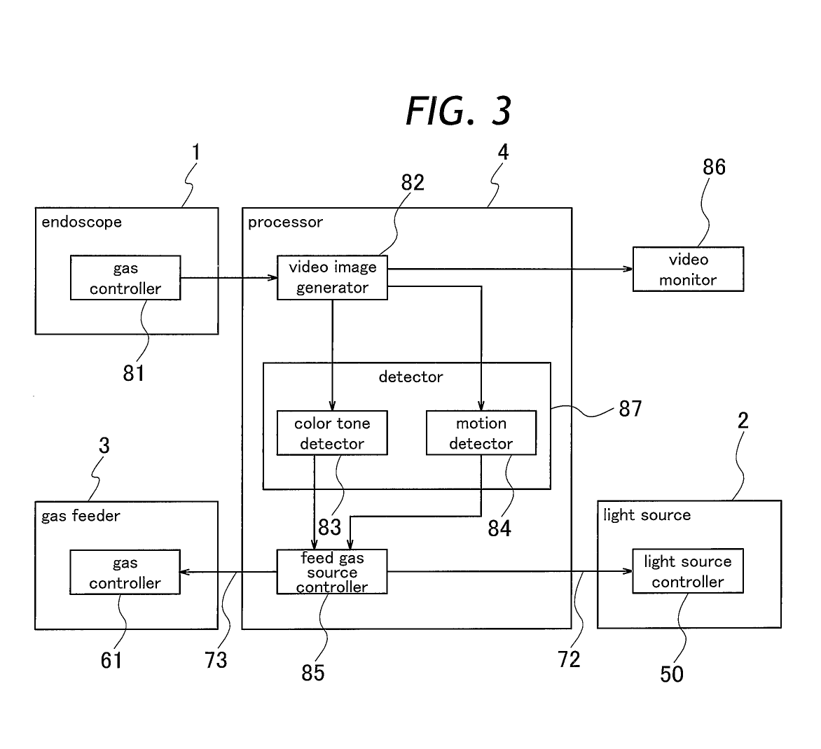

Schematically shown in FIG. 3 are configuration and connections between various components of the processor 4. By the solid-state image sensor device 81 of a camera unit at the fore distal end of the endoscopic insertion rod 11, incident light coming through the observation window 15 is converted into electric signals by photoelectric conversion. That is to say, images of a subject under observation at the fore distal end of the insertion rod 11 are captured and converted to electric signals, and the converted electric signals are output to the processor 4 via the light source connector 14.

The processor 4 is comprised of a video image generator 82, a color tone detector 83, a motion detector 84 and a feed gas source controller 85, and connected to a video monitor 86. At the video image generator 82, video signal data are generated on the basis of electric signals from the solid-state image sensor 81 by video signal processing operations known in the art. Picture images of an intracavitary site under observation by the endoscope can be viewed with eyes on a screen of the monitor 86 which is connected to the video image generator 82 to display generated video signal data.

The color tone detector 83 and motion detector 84 constitute a detector 87 which checks for a current status of an endoscope and make a judgment as to whether or not the endoscope 1 (more specifically, the insertion rod 11) is currently placed in a body cavity of a patient or examinee. Both of the color tone detector 83 and motion detector 84 operate on video signal data produced by the video image generator 82, but make judgments on the basis of different criteria.

The above-mentioned video signal data are data of picture images taken in the view field of the observation window 15 at the fore distal end of the insertion rod 11. That is to say, they are real time data of a picture image in the view field of the endoscope 1. Therefore, a current status of an endoscope, i.e. whether or not an endoscope 1 is currently placed in a body cavity, can be directly recognized from video signal data utilizing a certain aspect of the video signal data as a criterion. Namely, when in a body cavity, an intracavitary wall portion is taken in the view field of the endoscope 1, and a picture image of that intracavitary wall is captured as video signal data. On the other hand, when not inserted into a body cavity, for example, obtained video signal data are of a wall, floor or equipment in an endoscopy room which happens to fall in the view field of the observation window 15.

Picture images in a body cavity have a reddish color as a base color tone. On the other hand, picture images in an endoscopy room have a clearly different color tone or tones. Therefore, in case video signal data have a reddish color tone, it is recognized that they are data of a picture image captured in a body cavity. Otherwise, it is recognized that video signal data are of a picture image which is captured outside a body cavity. Accordingly, the color tone detector 83 is adapted to make an affirmative judgment that an endoscope is currently placed in a body cavity, when video signal data have a reddish color tone, and otherwise to make a negative judgment. The result of judgment is output to the feed gas source controller 85.

The motion detector 84 detects a motion of a subject from video signal data which are sequentially generated by the video signal generator 82, checking for a variation or variations which might have occurred between preceding and succeeding video signals as a result of a movement of a subject. Intracavitary walls are constantly put in motion by respiration of an examinee or due to peristalsis in the case of a digestive tract. Therefore, when in a body cavity, motions constantly take place in the view field of the observation window 15, causing variations in video signal data over time. Thus, when a motion of a subject is detected from video signal data, for example, this is recognized as a peristaltic motion which implies that an endoscope is currently placed in a body cavity. On the other hand, in case no motion of a subject is detected, it is judged that an endoscope is currently not placed in a body cavity. The result of judgment is output to the feed gas source controller 85.

The video image generator 82 performs predetermined video signal processing operations to convert electric signals to video signals. These operations are performed by a CPU which is provided in the processor 4 exclusively for this purpose, through utilization of image processing software. The detection of a color tone of video signals by the color tone detector 83 is effectuated by this image processing operation. Accordingly, functions of the color tone detector 83 can be performed by the use of a CPU and software which already exist in the video image generator 82.

The feed gas source controller 85 is adapted to control a feed gas source on the basis of judgment by both of or by either one of the color tone detector 83 and the motion detector 84. More particularly, it controls a carbon dioxide gas supply from the gas feeder 3 and an air supply from the light source 2 as well. For this purpose, the feed gas source controller 85 is connected to light source controller 50 and gas controller 61. However, as mentioned hereinbefore, it is preferable to use carbon dioxide gas as a fed gas source from the standpoint of lessening damages on the part of an examinee. Therefore, the feed gas controller 85 is adapted to preferentially supply carbon dioxide gas. As described hereinafter, an air supply from the light source 2 is limited to supplementary use.

With the arrangements as described above, it is possible to supply a gas and a liquid to the endoscope 1 by switching the fluid feed valve 20 to a gas feed position or a liquid feed position. For example, a liquid is supplied to the endoscope at the time of washing the observation window 1, while a gas or air is supplied at the time of dissipating liquid droplets from the observation window 15 after washing or at the time of inflating a body cavity. To make a liquid supply from the liquid feed tank 25, a pressure is applied to the surface of the liquid in the liquid feed tank 25 by introducing compressed air from the air pump 23 or carbon dioxide gas from the gas tank 60.

As mentioned hereinbefore, it has been the conventional practice to employ the air pump 23 solely as an air or gas source, permitting the fluid feed valve 20 to communicate with the atmosphere. In this case, the air pump 23 is constantly put in operation, and the pressure from the air pump 23 is released to the atmosphere at the time of idling. The communication with the atmosphere is blocked to start an air supply, manually switching the fluid feed valve 20 to start a liquid supply. The observation window 15 requires washing frequently, each time making a liquid supply in the first place to wash away contaminants and then switching to an air supply to blast compressed air for dissipation of liquid droplets from the observation window 15.

Recently, there is a trend toward use of carbon dioxide gas instead of air from the standpoint of lessening damages to a patient or examinee. However, since the fluid feed valve 20 is constantly communicated with the atmosphere unless a gas or liquid supply is started. Therefore, a supply of carbon dioxide gas from the gas tank 60 is kept on even when a gas or liquid supply to a body cavity is suspended, releasing a large quantity of carbon dioxide gas to the atmosphere, wastefully consuming carbon dioxide gas to a considerable degree.

Since the observation window 15 of the endoscope 1 requires washing frequently as mentioned above, arrangements should be made to permit a supply of carbon dioxide gas any time as long as the endoscope 1 is placed in a body cavity. On the other hand, the fluid feed valve 20 which is in communication with the atmosphere becomes a cause of consumption of a large quantity of carbon dioxide gas even when the endoscope 1 is not placed in a body cavity. Thus, the supply of carbon dioxide gas should be controlled in such a way that it is permitted only when the endoscope 1 is in a body cavity of a patient or examinee and suspended when not.

As the insertion rod 11 of the endoscope 1 is introduced into a body cavity, an intracavitary wall is taken in the view field of the endoscopic observation window 15, and video signal data are generated by the video signal generator 82 on the basis of electric signals from the solid-state image sensor device 81 to produce a video image of the intracavitary wall. Video signal data of an intracavitary image of this sort have a reddish color tone, and this is detected by the color tone detector 83. In addition, since the intracavitary wall can be in a peristaltic motion, and, if it is, that motion is detected by the motion detector 84. Accordingly, in this case, both of the color tone detector 83 and the motion detector 84 make an affirmative judgment that the endoscope 1 is currently placed in a body cavity.

The feed gas source controller 85 is connected to both of color tone detector 83 and motion detector 84, but it may be adapted to control a feed gas source on the basis of a judgment from one of the two detectors 83 and 84. Accordingly, if desired, either the color tone detector 83 or the motion detector 84 may be omitted. Of course, if desired, arrangements may be made to validate or invalidate judgments by one of the two detectors, the color tone detector 83 and the motion detector 84, which are connected to the feed gas source controller 85. Any way, the feed gas source controller 85 takes control of the gas controller 61 of the gas feeder 3 on the basis of a judgment or judgments of detectors.

Upon receiving an affirmative judgment that the endoscope 1 is currently placed in a body cavity, the feed gas source controller 85 controls the gas controller 61 through the second communication line 73 to fully open the valve 64 to supply carbon dioxide gas to the gas feed conduit 29. When the valve 64 is already open, the valve controller 61 controls the valve 64 simply to maintain the open state.

On the other hand, upon receipt of a negative judgment that the endoscope 1 is currently not placed in a body cavity, the gas controller 61 is controlled to close the valve 64. If the valve 64 is already closed, the gas controller 61 is controlled simply to maintain the closed state. In this regard, arrangements may be made to control the gas controller 61 by a 1-bit signal or to switch on and off a gas supply by such signal.

Accordingly, a carbon dioxide gas supply is allowed when it is ascertained that the endoscope 1 (the insertion rod 11) is currently placed in a body cavity, and suspended when the endoscope 1 outside a body cavity. Namely, depending upon the current status of the endoscope 1 (whether or not it is currently placed in a body cavity), the supply of carbon dioxide gas is automatically controlled, preventing suspension of a carbon dioxide gas supply while the endoscope 1 is in a body cavity, and suspending a carbon dioxide gas supply when not, to suppress wasteful consumption of carbon dioxide gas.

In case the feed gas source controller 81 is adapted to control a feed gas source on the basis of a judgment from one of the color tone detector 83 and the motion detector 84, it is desirable take preference of a judgment by the color tone detector 83. This is because, when the endoscope 1 is not inserted in a body cavity, a motion can be detected in the view field of the endoscope 1 which is gripped by an operator. Under circumstances like this, it is likely that a motion is detected in the view field of the endoscope 1, leading to a misjudgment that the endoscope 1 is currently placed in a body cavity. In this case, carbon dioxide gas is consumed wastefully through the endoscope 1 which is simply gripped in the hand of an operator outside a body cavity.

On the other hand, an endoscopy room has a clearly different color tone as compared with a body cavity, and it is very unlikely to detect an image of a reddish color tone when the endoscope 1 is not placed in a body cavity. Therefore, consumption of carbon dioxide gas can be suppressed effectively by preferentially relying on judgments by the color tone detector 83. In case judgments by the motion detector 84 are preferentially relied on, a supply of carbon dioxide gas is invariably feasible when the endoscope 1 is placed in a body cavity, by detection of peristaltic motions. However, in this case, the problem of wasteful consumption of carbon dioxide gas is solved only in a limited way. Thus, from the standpoint of safety, the preferential use of the motion detector 84 would not give rise to any problem in particular.

Further, arrangements may be made in such a way as to permit a supply of carbon dioxide gas only when both of the color tone detector 83 and the motion detector 84 make an affirmative judgment that the endoscope 1 is currently placed in a body cavity. Namely, in this case, a supply of carbon dioxide gas is permitted only when a reddish color tone is detected by the color tone detector 83 in addition to detection of a motion by the motion detector 84, otherwise suspending a gas supply. When both of the two detectors make an affirmative judgment, it is very sure that the endoscope 1 is currently placed in a body cavity. Thus, in this case, the status of the endoscope 1 can be judged in a very reliable manner.

Furthermore, arrangements may be made to control the gas supply by way of three steps or stages instead of two, including a stage-1 control which is put in effect when both of the color tone detector 83 and the motion detector 84 make a negative judgment, a stage-2 control which is put in effect when only one of the color tone detector 83 and the motion detector 84 makes an affirmative judgment, and a stage-3 control which is put in effect when both of the two detectors make an affirmative judgment. In this case, the feed gas source controller 85 is adapted to supply neither one of carbon dioxide gas and air through the endoscope 1 in stage-1 control, to supply only air in stage-2 control and to supply only carbon dioxide gas in stage-3 control.

In stage-1 control, that is, when both of the color tone detector 83 and the motion detector 84 make a negative judgment, a gas supply can be suspended safely. Therefore, the feed gas source controller 85 controls the gas controller 61 to stop a gas supply. At this time, a control signal may or may not be sent to the light source controller 50 to stop the air pump 23. If desired, the air pump 23 may be left in operation without any control because its operation does not involve the problem of wasteful consumption of carbon dioxide gas.

In stage-2 control, that is, different judgments are made by the color tone detector 83 and the motion detector 84. This occurs, for example, when the endoscope 1 gripped in a hand of an operator outside a patient's body cavity as mentioned hereinbefore. In this case, the feed gas source controller 85 suspends a carbon dioxide gas supply while permitting an air supply. Even if the endoscope 1 is placed in a body cavity at this time, air can be supplied thereto. Although a supply of carbon dioxide gas is preferable in consideration of its higher in vivo absorption, at least air is supplied to a body cavity to avoid the problem of total shutdown of gas supply. An air supply through an endoscope 1 which is inserted in a body cavity does not involve the problem of wasteful consumption of carbon dioxide gas.

In stage-3 control, by receipt of an affirmative judgment from both of the color tone detector 83 and the motion detector 84, it is double assured that the endoscope 1 is currently placed in a body cavity. Therefore, instead of air, a supply of carbon dioxide gas is permitted in this stage. By varying the gas supply mode in the three stages as described above, a supply of carbon dioxide gas, which is more desirable in consideration of in vivo absorption, is allowed only in a stage where it is ascertained that the endoscope 1 is currently placed in a body cavity in a quite assured manner, switching to an air supply in a stage where there is uncertainty as to the current status of the endoscope 1, precluding the danger of total shutdown of gas supply and wasteful consumption of carbon dioxide gas in a secure manner.

Further, the color tone detector 83 may be adapted to detect a reddish color tone of a captured video image in relation with its level in red color strength, e.g. ascertaining whether a detected reddish color is of a high red level or a low red level and changing the gas supply mode between a reddish color tone of a strong red level and a reddish color tone of a weak red level. For example, a strong red level and a weak red level can be detected from brightness of a red component in video signal data.

Among various reddish color tones detected, detection of a reddish color tone of a strong red level surely indicates that the endoscope 1 is currently placed in a body cavity. Accordingly, similarly to the stage-3 control described above, the feed gas source controller 85 permits a supply of carbon dioxide gas in this case. On the other hand, as compared with detection of a reddish color tone of a strong level, detection of a weak reddish color tone more or less lacks certainty as to the current status of the endoscope 1. Therefore, similarly to the stage-2 control described above, in this case the feed gas source controller 85 permits a supply of air. In case no reddish color tone is detected, the controller 85 inhibits a supply of carbon dioxide gas or air. By controlling the gas supply through the endoscope 1 in this manner, it becomes possible to avoid the danger of total shutdown of gas supply as well as wasteful consumption of carbon dioxide gas in an effective manner.

If desired, the motion detector 84 may be adapted to detect a motion of a subject on the basis of variations in brightness or luminance signal. The view field of the endoscope 1 changes as the insertion rod 11 is put in action, causing variations in intensity of light incident on the solid-state image sensor 81. These variations can be detected as variations in brightness signal. Accordingly, the motion detector 84 can be arranged to detect a motion of a subject from variations in brightness in video signal data. A motion of a subject can also be detected from a variation in opening degree of an aperture, and this can be utilized for detection of motion if desired.

In the foregoing embodiments, a color tone of a captured image is detected from video signal data, judging that an endoscope is currently placed in a body cavity upon detection of a reddish color tone. However, the present invention is not limited to this particular example. For instance, a reddish color tone can be used as a criterion color in observation under normal light, but arrangements may be made to judge the current status of an endoscope on the basis of other specific color in endoscopy under special light, spectral image processing endoscopy or narrow band filter endoscopy.

What is claimed is:

1. An endoscopic gaseous material feed system, comprising:
an endoscope having a solid-state image sensor device incorporated into a fore distal end portion of an elongated insertion rod to be placed in a body cavity of a patient or examinee;
a gas feeder having a gas tank packed with carbon dioxide gas to be supplied to said body cavity,
a light source incorporating an air pump to supply air to said body cavity;
a processor adapted to generate video signals of captured picture images on the basis electric signals from said solid-state image sensor;
a detector adapted to judge whether or not said endoscope is currently placed in a body cavity, on the basis of video signal data from said processor; and
a feed gas source controller adapted to permit a supply of carbon dioxide gas or air when said detector makes an affirmative judgment that said endoscope is currently placed in a body cavity and to prohibit a supply of carbon dioxide gas when said detector makes a negative judgment.

2. An endoscopic gaseous material feed system as set forth in claim 1, wherein said detector includes a color tone detector adapted to make an affirmative judgment that said endoscope is currently placed in a body cavity, upon detection of a reddish color tone in video signal data of an endoscopically captured picture image, and otherwise to make a negative judgment.

3. An endoscopic gaseous material feed system as set forth in claim 1, wherein said detector includes a motion detector adapted to make an affirmative judgment that said endoscope is currently placed in a body cavity, upon detection of a motion of a subject from video signal data of a captured picture image, and otherwise to make a negative judgment.

4. An endoscopic gaseous material feed system as set forth in claim 2, wherein said detector further includes a motion detector adapted to make an affirmative judgment that said endoscope is currently placed in a body cavity, upon detection of a motion of a subject from video signal data of a captured picture image, and otherwise to make a negative judgment, said feed gas source controller being adapted to control said gas feeder in such a way as to supply carbon dioxide gas in case an affirmative judgment is received from both of said color tone detector and said motion detector.

5. An endoscopic gaseous material feed system as set forth in claim 4, wherein said feed gas source controller is adapted to control said light source to supply air in case an affirmative judgment is received from only one of said color tone detector and said motion detector.

6. An endoscopic gaseous material feed system as set forth in claim 2, wherein said color tone detector is adapted to carry out a two-stage color tone detection, detecting a reddish color tone in video signal data of an endoscopically captured image in a first stage and checking whether or not a red color level of a detected reddish color tone is strong or weak in a second stage, said feed gas source controller being adapted to control said gas feeder to supply carbon dioxide gas in case a reddish color tone of a strong red level is detected by said color tone detector and to control said light source to supply air in case a reddish color tone of a weak red level is detected by said color tone detector.
